SEQUENCE LISTING (6 sequences)

SEQ ID NO: 1 - DNA, 4519 bp, Danio rerio
  CDS (191)...(2713)
agcagtggtaacaacgcagagtacgcggggctcgccgcctgaaataaaacccttccaagacttcttcaccaactttaaatccatcccaaggaatttactaatgggaaactgttaaaagagacccactttccgatggtggcccggatcagtttggcgttttatttattaggaagcccgcccggtcgcctttatgatggaaacgtgcgctcgccgtgtgcttttcacggccgctctgctcgtcctcagtactgtgatagcggaaactttctccacagactcggacaccgataatccactgagtacagaaactttttatagccgagcaagtggcttgaaacaaacctcctcctggccagggagagaggccactgccacagccgtggatctatcgagcggactgggagagatgacggagattcccgccagtgtatccatcactgcagcccgagagggacattcaccaaaacctttacaaacctctacaaatgcagcggattggaagaccagtacgacctcagatgagacaacagaacatcttcaatctgacactgagcttacccataatgcgaccgcccaatgggagagtccatcatcagcatctcacagcattaccagccaccatccagtaacagagacacgaaccgtgcgagatgtaacagatctgatagacatggacaccacagactcagtctcccacactgatagcacctacatttccacaaccaaccgagttggagaacgcacactgctctcagtgatctccaacagcacctttgcgtacacccagaactcaagcatctctgatgcagagtctcaaacgtccccatgggaggagaagacatcaggagccacccaagtcaatgaggaaactgaagaaactgtgtcaacagtgtccgaacagactgatcccacttttgaaggccgcaataccaccagtgcaactctggagactgagcggtcaacgttatcccaaggcactgagtcacagacaggacaacctagtgtcacaggacagaccgctaaggaggtgactgatatcgacaatccaaattcaacaccgcctcttacagtgaccagtagggatgttgaggaaacagatgccacatcagtgagcagtgagacatcttatacacaaaccagcagtgactctgcctcctcaatcttgccttttacttctagcgaacacaatgtcactagcacatcccaagagagccataattccacactcatctattccacaaatactggtggttccactgagttttcgactggatcagtaagctctactgcccatgaagaaactgaacggtcttcaactcgcatagtagatgagacaacccttcatgatgttacttctgcacccccagtgcttgaagatgtagccacaactatcgatgactcgctttccaagttcccttccggccaatcgcctaccatccctaaaaccgatgaccaaaccaacacacaagtggtgccaacatcaactcataggccacaggttacagatgaagccaccgatgaggtgtctacagtttacagttctaccactactttaactaccacaactccttctgtcaccactagacaactccaaccacactacaccacagtgcaaacccaaacacaacacactaccattgttaccaccgatatcattcaggtactgcgaacgacaccctctacagcccatcatgtgcctacattgactaccagtggaccacaggctccaagtacagctgattcttctgacgtcaccacattgcacttggaaaccagcacagccacgccggggaacactacggcgcacggtggacgtgcaacaacgccttttagcaagagcagcccgggtagaacaactgtggtagtgaccactggacatctcactgacaaaagcactacagagacaggaagcgcaaccacgcagatgcctcttagaacatcagcatcaccaggtcatgtatgtggacctaaaacctgtgcaaatggaggtcattgtgttagatcagctgaaggaagttactactgtcagtgtctctccgcatggaccggacccttctgcactgaagatgtggacgagtgtgtgaacagtccatgtcctcagggttcagtgtgtgtcaacacaggtggttctttcagctgtgaatgtgacctgggctttgacctggaggatggccgcagttgtacacaagtcaagacatttttgggcactttcacagtcaacaactctcttcatctcagaaatttaggtctgcacgagctacacagagagatccaacagctgctcaatgcttctctctccatcttccatggttatagacgctttaccctgggtaaaagagatggacaaggtgtgcaaatcccagtggtgagcatgttttcactctcctccaatgtgaccagcgcggatgttttcaacagcatccagatgtcccttaacaactgcagccggacatactcgcactgtcccattaaacttcagcaccagctctcctatcacgtggagagcctgtgcatggcccagaagaccaagtgtgatgtgcagtactcagattgctcggatattagcgggattcccaactgtcagtgccttcctgggtactttaaaaggaacccagaagacatgacctgcagagactgtggagatggactcaagcttgttaatggcaaatgtgtcgaatgcatgtttggatttggaggtttcaactgcaataattaaaagacaaaaacgacatcaacaagatcatcttcaaaagtggagagcttcagatgtcgccatacgcagagtttcctaagagcaatcgcgtgtccatggagtggggtagagagactatcgagatgcaggagaacggcagtacaaaaaacctcctgcaaatgactgacatttattactcgcctgcactgagaaactccgacctggagcgtaatggtctgtatccattctcgggccttcctggttcaaggcattcctgcatctatccggctcagtggaacccttccttcttaagcgacgattcacgccgaagagactacttttgacagctttacccagcaggcatgcaatcttgatgctgcatgtaagctctcttggcctgttgcagatcctgtagctcgattctgaccactaaaggcctttgaatgtgaaaatgagctggcaggaaaaggccattatctgtgagactggatgaaagccgcagagccctggcggggtcaatttagcattcggacaaagtgccacaaaaagattttagtatggagctgatgaaaggatgttcttcgctccccacagaccgcttattattaagcgcaagtcagcgaatttagactagccggaggaagaatacaaccacaagatgcctgagctacaatctaaaatttatattatgaaagtttgcactatgaacttctcacacagatattatttcaaatggagaaagccataaatgtatcatatttatacttggaatgatttacccagctggtttgtactgtgttaaggttttgtaaaggacaaaatatctggatgtttgtttgagtatgtgcatcaatagctgaagagataatttacccctagattttaattctgtcattatttactgtcgtacacggatgtccaaactcaatcctggagtgctggtgtcctgcaggctttagcttcaaccgtaattagacatcttaatcagctaaccaagctctatctaggccaggggtgctcaaccctgtacctggagataggaatggctgacatgaaactgacgtttcaacaccgtgtccagatcccgaaacactgcactgaagcatgatccgaaacacccaggtctcactggaagtactaggtcatccggagaagtacttctcgcatactgattttcgaattctttgaattcggacatactactcagctcacatactgatttttgcgtattgcatagtatggaagtatgcagttttggacgcagccacgatctctgcaagttagtcaggaactctcaccacttcactaaatgacttgaagcttcaactctacaacgtgaggtttgatacctcaatttggttaactttttcttcgctgaagctattccagagaaatacataaaaatgaccactaggtgtcactgtagagtggggtttctaaacgtttcgaagctttgaaacatttgctttgactgtttcagtgtttcatgaagcctcgctttgcccatcactacttggagatctaccttcttacagagttcagctccaaccctgatcaaacacacctgagccaattaattaggacctgaacagcactggataattacaggcaggtgactaggacacccctgatctaggcatactagaaacttccaggcaggtgtgttgaggcaagttagagctaaactatgcgggagtttgggcacctttgctctagtaatcctttgttaggagttgtttgttacaaaatgttcagctctttgctgtaaaatggtttaagaatcaagtttttaaagcataaaaaaaaaaaaaaaaaaaaaaaaaaaaa

SEQ ID NO: 2 - protein, 841 aa, Danio rerio
MMETCARRVLFTAALLVLSTVIAETFSTDSDTDNPLSTETFYSRASGLKQTSSWPGREATATAVDLSSGLGEMTEIPASVSITAAREGHSPKPLQTSTNAADWKTSTTSDETTEHLQSDTELTHNATAQWESPSSASHSITSHHPVTETRTVRDVTDLIDMDTTDSVSHTDSTYISTTNRVGERTLLSVISNSTFAYTQNSSISDAESQTSPWEEKTSGATQVNEETEETVSTVSEQTDPTFEGRNTTSATLETERSTLSQGTESQTGQPSVTGQTAKEVTDIDNPNSTPPLTVTSRDVEETDATSVSSETSYTQTSSDSASSILPFTSSEHNVTSTSQESHNSTLIYSTNTGGSTEFSTGSVSSTAHEETERSSTRIVDETTLHDVTSAPPVLEDVATTIDDSLSKFPSGQSPTIPKTDDQTNTQVVPTSTHRPQVTDEATDEVSTVYSSTTTLTTTTPSVTTRQLQPHYTTVQTQTQHTTIVTTDIIQVLRTTPSTAHHVPTLTTSGPQAPSTADSSDVTTLHLETSTATPGNTTAHGGRATTPFSKSSPGRTTVVVTTGHLTDKSTTETGSATTQMPLRTSASPGHVCGPKTCANGGHCVRSAEGSYYCQCLSAWTGPFCTEDVDECVNSPCPQGSVCVNTGGSFSCECDLGFDLEDGRSCTQVKTFLGTFTVNNSLHLRNLGLHELHREIQQLLNASLSIFHGYRRFTLGKRDGQGVQIPVVSMFSLSSNVTSADVFNSIQMSLNNCSRTYSHCPIKLQHQLSYHVESLCMAQKTKCDVQYSDCSDISGIPNCQCLPGYFKRNPEDMTCRDCGDGLKLVNGKCVECMFGFGGFNCNN

SEQ ID NO: 3 - protein, 977 aa, Danio rerio
MMETCARRVLFTAALLVLSTVIAETFSTDSDTDNPLSTETFYSRASGLKQTSSWPGREATATAVDLSSGLGEMTEIPASVSITAAREGHSPKPLQTSTNAADWKTSTTSDETTEHLQSDTELTHNATAQWESPSSASHSITSHHPVTETRTVRDVTDLIDMDTTDSVSHTDSTYISTTNRVGERTLLSVISNSTFAYTQNSSISDAESQTSPWEEKTSGATQVNEETEETVSTVSEQTDPTFEGRNTTSATLETERSTLSQGTESQTGQPSVTGQTAKEVTDIDNPNSTPPLTVTSRDVEETDATSVSSETSYTQTSSDSASSILPFTSSEHNVTSTSQESHNSTLIYSTNTGGSTEFSTGSVSSTAHEETERSSTRIVDETTLHDVTSAPPVLEDVATTIDDSLSKFPSGQSPTIPKTDDQTNTQVVPTSTHRPQVTDEATDEVSTVYSSTTTLTTTTPSVTTRQLQPHYTTVQTQTQHTTIVTTDIIQVLRTTPSTAHHVPTLTTSGPQAPSTADSSDVTTLHLETSTATPGNTTAHGGRATTPFSKSSPGRTTVVVTTGHLTDKSTTETGSATTQMPLRTSASPGHVCGPKTCANGGHCVRSAEGSYYCQCLSAWTGPFCTEDVDECVNSPCPQGSVCVNTGGSFSCECDLGFDLEDGRSCTQVKTFLGTFTVNNSLHLRNLGLHELHREIQQLLNASLSIFHGYRRFTLGKRDGQGVQIPVVSMFSLSSNVTSADVFNSIQMSLNNCSRTYSHCPIKLQHQLSYHVESLCMAQKTKCDVQYSDCSDISGVPNCQCLPGYFKRNPEDMTCRDCGDGLKLVNGKCVECMFGFGGFNCNNFYKLIAVVVSPAGGALLLIVVIALIVTCCKKDKNDINKIIFKSGELQMSPYAEFPKSNRVSMEWGRETIEMQENGSTKNLLQMTDIYYSPALRNSDLERNGLYPFSGLPGSRHSCIYPAQWNPSFLSDDSRRRDYF

SEQ ID NO: 4 - DNA, 4518 bp, Homo sapiens
  CDS (2)...(1963)
agcagtggtaacaacgcagagtacgcggggctcgccgcctgaaataaaacccttccaagacttcttcaccaactttaaatccatcccaaggaatttactaatgggaaactgttaaaagagacccactttccgatggtggcccggatcagtttggcgttttatttattaggaagcccgcccggtcgcctttatgatggaaacgtgcgctcgccgtgtgcttttcacggccgctctgctcgtcctcagtactgtgatagcggaaactttctccacagactcggacaccgataatccactgagtacagaaactttttatagccgagcaagtggcttgaaacaaacctcctcctggccagggagagaggccactgccacagccgtggatctatcgagcggactgggagagatgacggagattcccgccagtgtatccatcactgcagcccgagagggacattcaccaaaacctttacaaacctctacaaatgcagcggattggaagaccagtacgacctcagatgagacaacagaacatcttcaatctgacactgagcttacccataatgcgaccgcccaatgggagagtccatcatcagcatctcacagcattaccagccaccatccagtaacagagacacgaaccgtgcgagatgtaacagatctgatagacatggacaccacagactcagtctcccacactgatagcacctacatttccacaaccaaccgagttggagaacgcacactgctctcagtgatctccaacagcacctttgcgtacacccagaactcaagcatctctgatgcagagtctcaaacgtccccatgggaggagaagacatcaggagccacccaagtcaatgaggaaactgaagaaactgtgtcaacagtgtccgaacagactgatcccacttttgaaggccgcaataccaccagtgcaactctggagactgagcggtcaacgttatcccaaggcactgagtcacagacaggacaacctagtgtcacaggacagaccgctaaggaggtgactgatatcgacaatccaaattcaacaccgcctcttacagtgaccagtagggatgttgaggaaacagatgccacatcagtgagcagtgagacatcttatacacaaaccagcagtgactctgcctcctcaatcttgccttttacttctagcgaacacaatgtcactagcacatcccaagagagccataattccacactcatctattccacaaatactggtggttccactgagttttcgactggatcagtaagctctactgcccatgaagaaactgaacggtcttcaactcgcatagtagatgagacaacccttcatgatgttacttctgcacccccagtgcttgaagatgtagccacaactatcgatgactcgctttccaagttcccttccggccaatcgcctaccatccctaaaaccgatgaccaaaccaacacacaagtggtgccaacatcaactcataggccacaggttacagatgaagccaccgatgaggtgtctacagtttacagttctaccactactttaactaccacaactccttctgtcaccactagacaactccaaccacactacaccacagtgcaaacccaaacacaacacactaccattgttaccaccgatatcattcaggtactgcgaacgacaccctctacagcccatcatgtgcctacattgactaccagtggaccacaggctccaagtacagctgattcttctgacgtcaccacattgcacttggaaaccagcacagccacgccggggaacactacggcgcacggtggacgtgcaacaacgccttttagcaagagcagcccgggtagaacaactgtggtagtgaccactggacatctcactgacaaaagcactacagagacaggaagcgcaaccacgcagatgcctcttagaacatcagcatcaccaggtcatgtatgtggacctaaaacctgtgcaaatggaggtcattgtgttagatcagctgaaggaagttactactgtcagtgtctctccgcatggaccggacccttctgcactgaagatgtggacgagtgtgtgaacagtccatgtcctcagggttcagtgtgtgtcaacacaggtggttctttcagctgtgaatgtgacctgggctttgacctggaggatggccgcagttgtacacaagtcaagacatttttgggcactttcacagtcaacaactctcttcatctcagaaatttaggtctgcacgagctacacagagagatccaacagctgctcaatgcttctctctccatcttccatggttatagacgctttaccctgggtaaaagagatggacaaggtgtgcaaatcccagtggtgagcatgttttcactctcctccaatgtgaccagcgcggatgttttcaacagcatccagatgtcccttaacaactgcagccggacatactcgcactgtcccattaaacttcagcaccagctctcctatcacgtggagagcctgtgcatggcccagaagaccaagtgtgatgtgcagtactcagattgctcggatattagcgggattcccaactgtcagtgccttcctgggtactttaaaaggaacccagaagacatgacctgcagagactgtggagatggactcaagcttgttaatggcaaatgtgtcgaatgcatgtttggatttggaggtttcaactgcaataattaaagacaaaaacgacatcaacaagatcatcttcaaaagtggagagcttcagatgtcgccatacgcagagtttcctaagagcaatcgcgtgtccatggagtggggtagagagactatcgagatgcaggagaacggcagtacaaaaaacctcctgcaaatgactgacatttattactcgcctgcactgagaaactccgacctggagcgtaatggtctgtatccattctcgggccttcctggttcaaggcattcctgcatctatccggctcagtggaacccttccttcttaagcgacgattcacgccgaagagactacttttgacagctttacccagcaggcatgcaatcttgatgctgcatgtaagctctcttggcctgttgcagatcctgtagctcgattctgaccactaaaggcctttgaatgtgaaaatgagctggcaggaaaaggccattatctgtgagactggatgaaagccgcagagccctggcggggtcaatttagcattcggacaaagtgccacaaaaagattttagtatggagctgatgaaaggatgttcttcgctccccacagaccgcttattattaagcgcaagtcagcgaatttagactagccggaggaagaatacaaccacaagatgcctgagctacaatctaaaatttatattatgaaagtttgcactatgaacttctcacacagatattatttcaaatggagaaagccataaatgtatcatatttatacttggaatgatttacccagctggtttgtactgtgttaaggttttgtaaaggacaaaatatctggatgtttgtttgagtatgtgcatcaatagctgaagagataatttacccctagattttaattctgtcattatttactgtcgtacacggatgtccaaactcaatcctggagtgctggtgtcctgcaggctttagcttcaaccgtaattagacatcttaatcagctaaccaagctctatctaggccaggggtgctcaaccctgtacctggagataggaatggctgacatgaaactgacgtttcaacaccgtgtccagatcccgaaacactgcactgaagcatgatccgaaacacccaggtctcactggaagtactaggtcatccggagaagtacttctcgcatactgattttcgaattctttgaattcggacatactactcagctcacatactgatttttgcgtattgcatagtatggaagtatgcagttttggacgcagccacgatctctgcaagttagtcaggaactctcaccacttcactaaatgacttgaagcttcaactctacaacgtgaggtttgatacctcaatttggttaactttttcttcgctgaagctattccagagaaatacataaaaatgaccactaggtgtcactgtagagtggggtttctaaacgtttcgaagctttgaaacatttgctttgactgtttcagtgtttcatgaagcctcgctttgcccatcactacttggagatctaccttcttacagagttcagctccaaccctgatcaaacacacctgagccaattaattaggacctgaacagcactggataattacaggcaggtgactaggacacccctgatctaggcatactagaaacttccaggcaggtgtgttgaggcaagttagagctaaactatgcgggagtttgggcacctttgctctagtaatcctttgttaggagttgtttgttacaaaatgttcagctctttgctgtaaaatggtttaagaatcaagtttttaaagcataaaaaaaaaaaaaaaaaaaaaaaaaaaaa

SEQ ID NO: 5 - protein, 652 aa, Homo sapiens
AVVTTQSTRGSPPEIKPFQDFFTNFKSIPRNLLMGNCKRPTFRWWPGSVWRFIYEARPVAFMMETCARRVLFTAALLVLSTVIAETFSTDSDTDNPLSTETFYSRASGLKQTSSWPGREATATAVDLSSGLGEMTEIPASVSITAAREGHSPKPLQTSTNAADWKTSTTSDETTEHLQSDTELTHNATAQWESPSSASHSITSHHPVTETRTVRDVTDLIDMDTTDSVSHTDSTYISTTNRVGERTLLSVISNSTFAYTQNSSISDAESQTSPWEEKTSGATQVNEETEETVSTVSEQTDPTFEGRNTTSATLETERSTLSQGTESQTGQPSVTGQTAKEVTDIDNPNSTPPLTVTSRDVEETDATSVSSETSYTQTSSDSASSILPFTSSEHNVTSTSQESHNSTLIYSTNTGGSTEFSTGSVSSTAHEETERSSTRIVDETTLHDVTSAPPVLEDVATTIDDSLSKFPSGQSPTIPKTDDQTNTQVVPTSTHRPQVTDEATDEVSTVYSSTTTLTTTTPSVTTRQLQPHYTTVQTQTQHTTIVTTDIIQVLRTTPSTAHHVPTLTTSGPQAPSTADSSDVTTLHLETSTATPGNTTAHGGRATTPFSKSSPGRTTVVVTTGHLTDKSTTETGSATTQMPLRTSASPGHVC

SEQ ID NO: 6 - protein, 654 aa, Homo sapiens
SAPLSVSQTTLPQSSSTPVLPRARETPVTSFQTSTMTSFMTMLHSSQTADLKSQSTPHQEKVITESKSPSLVSLPTESTKAVTTNSPLPPSLTESSTEQTLPATSTNLAQMSPTFTTTILKTSQPLMTTPGTLSSTASLVTGPIAVQTTAGKQLSLTHPEILVPQISTEGGISTERNRVIVDATTGLIPLTSVPTSAKEMTTKLGVTAEYSPASRSLGTSPSPQTTVVSTAEDLAPKSATFAVQSSTQSPTTLSSSASVNSCAVNPCLHNGECVADNTSRGYHCRCPPSWQGDDCSVDVNECLSNPCPSTATCNNTQGSFICKCPVGYQLEKGICNLVRTFVTEFKLKRTFLNTTVEKHSDLQEVENEITKTLNMCFSALPSYIRSTVHASRESNAVVISLQTTFSLASNVTLFDLADRMQKCVNSCKSSAEVCQLLGSQRRIFRAGSLCKRKSPECDKDTSICTDLDGVALCQCKSGYFQFNKMDHSCRACEDGYRLENETCMSCPFGLGGLNCGNPYQLITVVIAAAGGGLLLILGIALIVTCCRKNKNDISKLIFKSGDFQMSPYAEYPKNPRSQEWGREAIEMHENGSTKNLLQMTDVYYSPTSVRNPELERNGLYPAYTGLPGSRHSCIFPGQYNPSFISDESRRRDYF

(19) United States
(12) Patent Application Publication
(10) Pub. No.: US 2009/0077681 A1
(43) Pub. Date: Mar. 19, 2009

(54) Methods for Diagnosing and Treating Heart Disease

(75) Inventors: Mark C. Fishman, Newton, MA (US); John Mably, Medford, MA (US)

Correspondence Address: CLARK & ELBING LLP, 101 FEDERAL STREET, BOSTON, MA, 02110, US

(73) Assignee: The General Hospital Corporation, Boston, MA (US)

(21) Appl. No.: 12/115,986

(22) Filed: May 6, 2008

Related U.S. Application Data
(63) Continuation of application No. 10/467,490, filed on Mar. 3, 2004, now abandoned.
(60) Provisional application No. 60/267,334, filed on Feb. 7, 2001.

(51) Int. Cl. C12Q 1/68; C07K 14/435; C07H 21/00; C12N 15/63; C12N 5/06; A01K 67/027; C07K 16/00

(52) U.S. Cl. 800/20; 435/6; 530/350; 536/23.5; 435/320.1; 435/325; 530/387.9

(57) ABSTRACT

The invention provides methods of diagnosing heart disease, such as heart failure, methods for identifying compounds that can be used to treat or to prevent heart disease, and methods of using these compounds to treat or to prevent heart disease. Also provided in the invention are animal model systems that can be used in screening methods.

### FIELD OF THE INVENTION

This invention relates to methods for diagnosing and treating heart disease.

### BACKGROUND OF THE INVENTION

Heart disease is a general term used to describe many different heart conditions. For example, coronary artery disease, which is the most common heart disease, is characterized by constriction or narrowing of the arteries supplying the heart with oxygen-rich blood, and can lead to myocardial infarction, which is the death of a portion of the heart muscle. Heart failure is a condition resulting from the inability of the heart to pump an adequate amount of blood through the body. Heart failure is not a sudden, abrupt stop of heart activity but, rather, typically develops slowly over many years, as the heart gradually loses its ability to pump blood efficiently. Risk factors for heart failure include coronary artery disease, hypertension, valvular heart disease, cardiomyopathy, disease of the heart muscle, obesity, diabetes, and a family history of heart failure.

### SUMMARY OF THE INVENTION

The invention provides diagnostic, drug screening, and therapeutic methods that are based on the observation that a mutation in a zebrafish gene, designated heart of glass (heg), leads to a phenotype in zebrafish that is similar to heart failure in mammals.

In a first aspect, the invention provides a method of determining whether a test subject (e.g., a mammal, such as a human) has, or is at risk of developing, a disease or condition related to a heart of glass protein (e.g., heart disease, such as heart failure). This method involves analyzing a nucleic acid molecule of a sample from the test subject to determine whether the test subject has a mutation (e.g., the heart of glass mutation) in a gene encoding the protein. The presence of a mutation indicates that the test subject has, or is at risk of developing, a disease related to a heart of glass protein. This method can also involve the step of using nucleic acid molecule primers specific for a gene encoding a heart of glass protein for nucleic acid molecule amplification of the gene by the polymerase chain reaction. It can also involve sequencing a nucleic acid molecule encoding a heart of glass protein from said test subject.

In a second aspect, the invention provides a method for identifying a compound that can be used to treat or to prevent heart disease (e.g., heart failure). This method involves contacting an organism (e.g., a zebrafish) having a mutation (e.g., the heart of glass mutation) in a gene encoding a heart of glass protein and having a phenotype characteristic of heart disease with the compound, and determining the effect of the compound on the phenotype. Detection of an improvement in the phenotype indicates the identification of a compound that can be used to treat or to prevent heart disease.

In a third aspect, the invention provides a method of treating or preventing heart disease (e.g., heart failure) in a patient (e.g., a patient having a mutation (e.g., the heart of glass mutation) in a gene encoding a heart of glass protein), involving administering to the patient a compound identified using the method described above.

In a fourth aspect, the invention provides of treating or preventing heart disease in a patient. This method involves administering to the patient a functional heart of glass protein or an expression vector including a nucleic acid molecule encoding the protein.

In a fifth aspect, the invention includes a substantially pure zebrafish heart of glass polypeptide. This polypeptide can include or consist essentially of, for example, an amino acid sequence that is substantially identical to the amino acid sequence of SEQ ID NO:2 or SEQ ID NO:3.

In a sixth aspect, the invention provides a substantially pure nucleic acid molecule (e.g., a DNA molecule) including a sequence encoding a zebrafish heart of glass polypeptide. This nucleic acid molecule can encode a polypeptide including or consisting essentially of an amino sequence that is substantially identical to the amino acid sequence of SEQ ID NO:2 or SEQ ID NO:3.

In a seventh aspect, the invention provides a vector including the nucleic acid molecule described above.

In an eighth aspect, the invention includes a cell including the vector described above.

In a ninth aspect, the invention provides a non-human transgenic animal (e.g., a zebrafish) including the nucleic acid molecule described above.

In a tenth aspect, the invention provides a non-human animal having a knockout mutation in one or both alleles encoding a heart of glass polypeptide.

In an eleventh aspect, the invention includes a cell from the non-human knockout animal described above.

In a twelfth aspect, the invention includes a non-human transgenic animal (e.g., a zebrafish) including a nucleic acid molecule encoding a mutant heart of glass polypeptide, e.g., a polypeptide having the heart of glass mutation.

In a thirteenth aspect, the invention provides an antibody that specifically binds to a heart of glass polypeptide.

In a fourteenth aspect, the invention provides the use of a compound identified using the method described above in the preparation of a medicament for treating or preventing heart disease in a patient.

In a fifteenth aspect, the invention provides the use of a heart of glass protein or an expression vector including a nucleic acid molecule encoding this protein in the preparation of a medicament for treating or preventing heart disease in a patient.

By “polypeptide” or “polypeptide fragment” is meant a chain of two or more amino acids, regardless of any post-translational modification (e.g., glycosylation or phosphorylation), constituting all or part of a naturally or non-naturally occurring polypeptide. By “post-translational modification” is meant any change to a polypeptide or polypeptide fragment during or after synthesis. Post-translational modifications can be produced naturally (such as during synthesis within a cell) or generated artificially (such as by recombinant or chemical means). A “protein” can be made up of one or more polypeptides.

By “heart of glass protein” or “heart of glass polypeptide” is meant a polypeptide that has at least 45%, preferably at least 60%, more preferably at least 75%, and most preferably at least 90% amino acid sequence identity to the sequence of a human (see, e.g., SEQ ID NO:5) or a zebrafish (see, e.g., SEQ ID NO:2 or SEQ ID NO:3) heart of glass polypeptide. Polypeptide products from splice variants (e.g., heg1 and heg2) of heart of glass gene sequences and heart of glass genes containing mutations are also included in this definition. A heart of glass polypeptide as defined herein plays a role in heart development, modeling, and function. It can be used as a marker of heart disease, such as heart failure. The invention thus includes proteins having any of these and other functions of heart of glass, as described herein, and having sequence identity (e.g., at least 75%, 85%, 90%, or 95%) to a human or a zebrafish (SEQ ID NO:2 or SEQ ID NO:3) heart of glass polypeptide.

By a “heart of glass nucleic acid molecule” is meant a nucleic acid molecule, such as a genomic DNA, cDNA, or RNA (e.g., mRNA) molecule, that encodes a heart of glass protein (e.g., a human (encoded by, e.g., SEQ ID NO:4) or a zebrafish (encoded by, e.g., SEQ ID NO:1) heart of glass protein), a heart of glass polypeptide, or a portion thereof, as defined above. A mutation in a heart of glass nucleic acid molecule can be characterized, for example, by a G to A nucleotide transversion at position 497, predicting a change from a tryptophan codon to a stop codon (TGG->TAG). In addition to these zebrafish heart of glass mutations, the invention includes any mutation that results in aberrant heart of glass protein production or function, including, only as examples, null mutations and mutations causing truncations.

The term “identity” is used herein to describe the relationship of the sequence of a particular nucleic acid molecule or polypeptide to the sequence of a reference molecule of the same type. For example, if a polypeptide or a nucleic acid molecule has the same amino acid or nucleotide residue at a given position, compared to a reference molecule is to which it is aligned, there is said to be “identity” at that position. The level of sequence identity of a nucleic acid molecule or a polypeptide to a reference molecule is typically measured using sequence analysis software with the default parameters specified therein, such as the introduction of gaps to achieve an optimal alignment (e.g., Sequence Analysis Software Package of the Genetics Computer Group, University of Wisconsin Biotechnology Center, 1710 University Avenue, Madison, Wis. 53705, BLAST, or PILEUP/PRETTYBOX programs). These software programs match identical or similar sequences by assigning degrees of identity to various substitutions, deletions, or other modifications. Conservative substitutions typically include substitutions within the following groups: glycine, alanine, valine, isoleucine, and leucine; aspartic acid, glutamic acid, asparagine, and glutamine; serine and threonine; lysine and arginine; and phenylalanine and tyrosine.

A nucleic acid molecule or polypeptide is said to be “substantially identical” to a reference molecule if it exhibits, over its entire length, at least 51%, preferably at least 55%, 60%, or 65%, and most preferably 75%, 85%, 90%, or 95% identity to the sequence of the reference molecule. For polypeptides, the length of comparison sequences is at least 16 amino acids, preferably at least 20 amino acids, more preferably at least 25 amino acids, and most preferably at least 35 amino acids. For nucleic acid molecules, the length of comparison sequences is at least 50 nucleotides, preferably at least 60 nucleotides, more preferably at least 75 nucleotides, and most preferably at least 110 nucleotides.

A heart of glass nucleic acid molecule or a heart of glass polypeptide is “analyzed” or subject to “analysis” if a test procedure is carried out on it that allows the determination of its biological activity or whether it is wild type or mutated. For example, one can analyze the heart of glass genes of an animal (e.g., a human or a zebrafish) by amplifying genomic DNA of the animal using the polymerase chain reaction, and then determining whether the amplified DNA contains a mutation, for example, the heart of glass mutation, by, e.g., nucleotide sequence or restriction fragment analysis.

By “probe” or “primer” is meant a single-stranded DNA or RNA molecule of defined sequence that can base pair to a second DNA or RNA molecule that contains a complementary sequence (“target”). The stability of the resulting hybrid depends upon the extent of the base pairing that occurs. This stability is affected by parameters such as the degree of complementarity between the probe and target molecule, and the degree of stringency of the hybridization conditions. The degree of hybridization stringency is affected by parameters such as the temperature, salt concentration, and concentration of organic molecules, such as formamide, and is determined by methods that are well known to those skilled in the art. Probes or primers specific for heart of glass nucleic acid molecules, preferably, have greater than 45% sequence identity, more preferably at least 55-75% sequence identity, still more preferably at least 75-85% sequence identity, yet more preferably at least 85-99% sequence identity, and most preferably 100% sequence identity to the sequences of human or zebrafish (SEQ ID NO:1) heart of glass genes (either strand). Preferably, the probes or primers bind to heart of glass genes (or the complements thereof) under highly stringent conditions, as described herein.

Probes can be detectably labeled, either radioactively or non-radioactively, by methods that are well known to those skilled in the art. Probes can be used for methods involving nucleic acid hybridization, such as nucleic acid sequencing, nucleic acid amplification by the polymerase chain reaction, single stranded conformational polymorphism (SSCP) analysis, restriction fragment polymorphism (RFLP) analysis, Southern hybridization, northern hybridization, in situ hybridization, electrophoretic mobility shift assay (EMSA), and other methods that are well known to those skilled in the art.

A molecule, e.g., an oligonucleotide probe or primer, a gene or fragment thereof, a cDNA molecule, a polypeptide, or an antibody, can be said to be “detectably-labeled” if it is marked in such a way that its presence can be directly identified in a sample. Methods for detectably labeling molecules are well known in the art and include, without limitation, radioactive labeling (e.g., with an isotope, such as ³²P or ³⁵S) and nonradioactive labeling (e.g., with a fluorescent label, such as fluorescein).

By a “substantially pure polypeptide” is meant a polypeptide (or a fragment thereof) that has been separated from proteins and organic molecules that naturally accompany it. Typically, a polypeptide is substantially pure when it is at least 60%, by weight, free from the proteins and naturally occurring organic molecules with which it is naturally associated. Preferably, the polypeptide is a heart of glass polypeptide that is at least 75%, more preferably at least 90%, and most preferably at least 99%, by weight, pure. A substantially pure heart of glass polypeptide can be obtained, for example, by extraction from a natural source (e.g., isolated heart tissue), by expression of a recombinant nucleic acid molecule encoding a heart of glass polypeptide, or by chemical synthesis. Purity can be measured by any appropriate method, e.g., by column chromatography, polyacrylamide gel electrophoresis, or HPLC analysis.

A polypeptide is substantially free of naturally associated components when it is separated from those proteins and organic molecules that accompany it in its natural state. Thus, a protein that is chemically synthesized or produced in a cellular system that is different from the cell in which it is naturally produced is substantially free from its naturally associated components. Accordingly, substantially pure polypeptides not only include those that are derived from eukaryotic organisms, but also those synthesized in E. coli or other prokaryotes.

An antibody is said to “specifically bind” to a polypeptide if it recognizes and binds to the polypeptide (e.g., a heart of glass polypeptide), but does not substantially recognize and bind to other molecules (e.g., non-heart of glass related polypeptides) in a sample, e.g., a biological sample that naturally includes the polypeptide.

By “high stringency conditions” is meant conditions that allow hybridization comparable with the hybridization that occurs using a DNA probe of at least 500 nucleotides in length, in a buffer containing 0.5 M NaHPO₄, pH 7.2, 7% SDS, 1 mM EDTA, and 1% BSA (fraction V), at a temperature of 65° C., or a buffer containing 48% formamide, 4.8×SSC, 0.2 M Tris-Cl, pH 7.6, 1×Denhardt's solution, 10% dextran sulfate, and 0.1% SDS, at a temperature of 42° C. (These are typical conditions for high stringency northern or Southern hybridizations.) High stringency hybridization is also relied upon for the success of numerous techniques routinely performed by molecular biologists, such as high stringency PCR, DNA sequencing, single strand conformational polymorphism analysis, and in situ hybridization. In contrast to northern and Southern hybridizations, these techniques are usually performed with relatively short probes (e.g., usually 16 nucleotides or longer for PCR or sequencing, and 40 nucleotides or longer for in situ hybridization). The high stringency conditions used in these techniques are well known to those skilled in the art of molecular biology, and examples of them can be found, for example, in Ausubel et al., Current Protocols in Molecular Biology, John Wiley & Sons, New York, N.Y., 1998, which is hereby incorporated by reference.

By “sample” is meant a tissue biopsy, amniotic fluid, cell, blood, serum, urine, stool, or other specimen obtained from a patient or a test subject. The sample can be analyzed to detect a mutation in a heart of glass gene, or expression levels of a heart of glass gene, by methods that are known in the art. For example, methods such as sequencing, single-strand conformational polymorphism (SSCP) analysis, or restriction fragment length polymorphism (RFLP) analysis of PCR products derived from a patient sample can be used to detect a mutation in a heart of glass gene; ELISA can be used to measure levels of a heart of glass polypeptide; and PCR can be used to measure the level of a heart of glass nucleic acid molecule.

By “heart of glass-related disease” or “heart of glass-related condition” is meant a disease or condition that results from inappropriately high or low expression of a heart of glass gene, or a mutation in a heart of glass gene that alters the biological activity of a heart of glass nucleic acid molecule or polypeptide. Heart of glass-related diseases and conditions can arise in any tissue in which heart of glass is expressed during prenatal or post-natal life. Heart of glass-related diseases and conditions can include heart diseases, such as heart failure.

The invention provides several advantages. For example, using the diagnostic methods of the invention it is possible to detect an increased likelihood of heart disease, such as heart failure, in a patient, so that appropriate intervention can be instituted before any symptoms occur. This may be useful, for example, with patients in high-risk groups for heart failure. Also, the diagnostic methods of the invention facilitate determination of the etiology of an existing heart condition, such as heart failure, in a patient so that an appropriate approach to treatment can be selected. In addition, the screening methods of the invention can be used to identify compounds that can be used to treat or to prevent heart conditions, such as heart failure.

Other features and advantages of the invention will be apparent from the following detailed description, the drawings, and the claims.

### BRIEF DESCRIPTION OF THE DRAWINGS

FIG. 1 is a schematic representation of a map of the heg interval. YAC clones are indicated by the addresses beginning with “b.” The BAC clone 110e08 contains recombinants on either side of the mutation and was sequenced to determine the heg gene.

FIG. 2 is a schematic representation of the genomic structure of the heg gene. The gene comprises 22,000 basepairs of genomic sequence, not including the 5′ upstream region, and encodes an mRNA of 4.5 kb in length.

FIG. 3 is a schematic representation of an alignment of the heg gene with kiaa1237 (Genbank entry BAA86551.1). The cDNA sequence shown for heg1 is derived from ligation of an oligo(dT)-primed 2.5 kb cDNA clone to a 2 kb 5′RACE product, and represents a putative secreted form. Heg2 is based on sequence of a PCR product using primers surrounding the stop codon in heg1 and encodes a potential membrane spanning domain and a highly conserved intracellular domain. The putative signal peptide and EGF-repeats are indicated with hashed lines. The premature stop associated with the heg mutation is indicated by an asterisk.

### DETAILED DESCRIPTION

The invention provides methods of diagnosing heart disease, screening methods for identifying compounds that can be used to treat or to prevent heart disease, and methods of treating or preventing heart disease using such compounds. In particular, we have discovered that a mutation (the heart of glass mutation) in a zebrafish gene, designated herein as heart of glass, leads to a phenotype in zebrafish that is similar to heart failure in mammals. Thus, the diagnostic methods of the invention involve detection of mutations in genes encoding heart of glass proteins, while the compound identification methods involve screening for compounds that affect the phenotype of organisms having mutations in genes encoding such proteins or other models of heart failure. Compounds identified in this manner can be used in methods to treat or to prevent heart disease, such as heart failure.

The invention also provides animal model systems (e.g., zebrafish having mutations (e.g., the heart of glass mutation) in genes encoding the heart of glass protein, or mice (or other animals) having such mutations) that can be used in the screening methods mentioned above, as well as the heart of glass protein, and genes encoding this protein. Also included in the invention are genes encoding mutant zebrafish heart of glass proteins (e.g., genes having the heart of glass mutation) and proteins encoded by these genes. Antibodies that specifically bind to these proteins (wild type or mutant) are also included in the invention.

The diagnostic, screening, and therapeutic methods of the invention, as well as the animal model systems, proteins, and genes of the invention, are described further, as follows.

### Diagnostic Methods

Nucleic acid molecules encoding the heart of glass protein, as well as polypeptides encoded by these nucleic-acid molecules and antibodies specific for these polypeptides, can be used in methods to diagnose or to monitor diseases and conditions involving mutations in, or inappropriate expression of, genes encoding this protein. As discussed above, the heart of glass mutation in zebrafish is characterized by a phenotype that is similar to that of heart failure in mammals, such as humans. Thus, detection of abnormalities in heart of glass genes or in their expression can be used in methods to diagnose, or to monitor treatment or development of, human heart disease, such as heart failure.

The diagnostic methods of the invention can be used, for example, with patients that have heart failure, in an effort to determine its etiology and, thus, to facilitate selection of an appropriate course of treatment. The diagnostic methods can also be used with patients who have not yet developed heart failure, but who are at risk of developing such a disease, or with patients that are at an early stage of developing such a disease. Also, the diagnostic methods of the invention can be used in prenatal genetic screening, for example, to identify parents who may be carriers of a recessive mutation in a gene encoding a heart of glass protein.

Examples of heart failure that can be diagnosed (and treated) using the methods of the invention include congestive heart failure, which is characterized by fluid in the lungs or body, resulting from failure of the heart in acting as a pump; right sided heart failure (right ventricular), which is characterized by failure of the pumping action of the right ventricle, resulting in swelling of the body, especially the legs and abdomen; left sided heart failure (left ventricular), which is caused by failure of the pumping action of the left side of the heart, resulting in congestion of the lungs; forward heart failure, which is characterized by the inability of the heart to pump blood forward at a sufficient rate to meet the oxygen needs of the body at rest or during exercise; backward heart failure, which is characterized by the ability of the heart to meet the needs of the body only if heart filling pressures are abnormally high; low-output, which is characterized by failure to maintain blood output; and high-output, which is characterized by heart failure symptoms, even when cardiac output is high.

Heart of glass may also play a role in cardiovascular diseases other than heart failure, such as coronary artery disease or conditions associated with valve formation defects, and, thus, detection of abnormalities in heart of glass genes or their expression can be used in methods to diagnose and monitor these conditions as well. The methods of the invention can be used to diagnose (or to treat) the disorders described herein in any mammal, for example, humans, domestic pets, or livestock.

Abnormalities in heart of glass that can be detected using the diagnostic methods of the invention include those characterized by, for example, (i) a gene encoding a heart of glass protein containing a mutation that results in the production of an abnormal heart of glass protein, (ii) an abnormal heart of glass polypeptide itself, and (iii) a mutation in a gene encoding a heart of glass protein that results in production of an abnormal amount of this protein. Detection of such abnormalities can be used in methods to diagnose human heart disease, such as heart failure (see above). Exemplary of the mutations in a heart of glass protein is the heart of glass mutation, which is described further below.

A mutation in a gene encoding a heart of glass protein can be detected in any tissue of a subject, even one in which this protein is not expressed. Because of the limited number of tissues in which these proteins are expressed (e.g., the myocardium, neurons, and smooth muscle) and because of the undesirability of sampling such tissues for assays, it may be preferable to detect mutant genes in other, more easily obtained sample types, such as in blood or amniotic fluid samples.

Detection of a mutation in a gene encoding a heart of glass protein can be carried out using any standard diagnostic technique. For example, a biological sample obtained from a patient can be analyzed for one or more mutations (e.g., a heart of glass mutation; see below) in nucleic acid molecules encoding a heart of glass protein using a mismatch detection approach. Generally, this approach involves polymerase chain reaction (PCR) amplification of nucleic acid molecules from a patient sample, followed by identification of a mutation (i.e., a mismatch) by detection of altered hybridization, aberrant electrophoretic gel migration, binding, or cleavage mediated by mismatch binding proteins, or by direct nucleic acid molecule sequencing. Any of these techniques can be used to facilitate detection of a mutant gene encoding a heart of glass protein, and each is well known in the art. For instance, examples of these techniques are described by Orita et al. (Proc. Natl. Acad. Sci. U.S.A. 86:2766-2770, 1989) and Sheffield et al. (Proc. Natl. Acad. Sci. U.S.A. 86:232-236, 1989).

In addition to facilitating diagnosis of existing heart disease, mutation detection assays also provide an opportunity to diagnose a predisposition to heart disease related to a mutation in a gene encoding a heart of glass protein before the onset of symptoms. For example, a patient who is heterozygous for a gene encoding an abnormal heart of glass protein (or an abnormal amount thereof) that suppresses normal heart of glass biological activity or expression may show no clinical symptoms of a disease related to such proteins, and yet possess a higher than normal probability of developing heart disease, such as heart failure. Given such a diagnosis, a patient can take precautions to minimize exposure to adverse environmental factors, and can carefully monitor their medical condition, for example, through frequent physical examinations. As mentioned above, this type of diagnostic approach can also be used to detect a mutation in a gene encoding the heart of glass protein in prenatal screens.

While it may be preferable to carry out diagnostic methods for detecting a mutation in a gene encoding the heart of glass protein using genomic DNA from readily accessible tissues, mRNA encoding this protein, or the protein itself, can also be assayed from tissue samples in which it is expressed, and may not be so readily accessible. For example, expression levels of a gene encoding the heart of glass protein in such a tissue sample from a patient can be determined by using any of a number of standard techniques that are well known in the art, including northern blot analysis and quantitative PCR (see, e.g., Ausubel et al., supra; PCR Technology: Principles and Applications for DNA Amplification, H. A. Ehrlich, Ed., Stockton Press, NY; Yap et al. Nucl. Acids. Res. 19:4294, 1991).

In another diagnostic approach of the invention, an immunoassay is used to detect or to monitor the level of a heart of glass protein in a biological sample. Polyclonal or monoclonal antibodies specific for the heart of glass protein can be used in any standard immunoassay format (e.g., ELISA, Western blot, or RIA; see, e.g., Ausubel et al., supra) to measure polypeptide levels of the heart of glass protein. These levels can be compared to levels of the heart of glass protein in a sample from an unaffected individual. Detection of a decrease in production of the heart of glass protein using this method, for example, may be indicative of a condition or a predisposition to a condition involving insufficient biological activity of the heart of glass protein.

Immunohistochemical techniques can also be utilized for detection of the heart of glass protein in patient samples. For example, a tissue sample can be obtained from a patient, sectioned, and stained for the presence of the heart of glass protein using an anti-heart of glass protein antibody and any standard detection system (e.g., one that includes a secondary antibody conjugated to an enzyme such as horseradish peroxidase). General guidance regarding such techniques can be found in, e.g., Bancroft et al., Theory and Practice of Histological Techniques, Churchill Livingstone, 1982, and Ausubel et al., supra.

Identification of Molecules that can be Used to Treat or to Prevent Heart Failure

Identification of a mutation in the heart of glass gene encoding the heart of glass protein as resulting in a phenotype that is related to heart failure facilitates the identification of molecules (e.g., small organic or inorganic molecules, peptides, or nucleic acid molecules) that can be used to treat or to prevent heart disease, such as heart failure. The effects of candidate compounds on heart failure can be investigated using, for example, the zebrafish system. The zebrafish, Danio rerio, is a convenient organism to use in genetic analysis of vascular development. In addition to its short generation time and fecundity, it has an accessible and transparent embryo, allowing direct observation of blood vessel function from the earliest stages of development. As discussed further below, zebrafish and other animals having a heart of glass mutation, which can be used in these methods, are also included in the invention.

In one example of the screening methods of the invention, a zebrafish having a mutation in a gene encoding the heart of glass protein (e.g., a zebrafish having the heart of glass mutation) is contacted with a candidate compound, and the effect of the compound on the development of a heart abnormality that is characteristic of heart failure, or on the status of such an existing heart abnormality, is monitored relative to an untreated, identically mutant control. As discussed further below, zebrafish having the heart of glass mutation are characterized by an enlarged and distended heart. Thus, these characteristics (in addition to other characteristics of heart disease) can be monitored using the screening methods of the invention.

After a compound has been shown to have a desired effect in the zebrafish system, it can be tested in other models of heart disease, for example, in mice or other animals having a mutation in a gene encoding the heart of glass protein. Alternatively, testing in such animal model systems can be carried out in the absence of zebrafish testing.

Cell culture-based assays can also be used in the identification of molecules that increase or decrease heart of glass levels or biological activity. According to one approach, candidate molecules are added at varying concentrations to the culture medium of cells expressing heart of glass mRNA. Heart of glass biological activity is then measured using standard techniques. The measurement of biological activity can include the measurement of heart of glass protein and nucleic acid molecule levels.

In general, novel drugs for prevention or treatment of heart diseases related to mutations in a gene encoding the heart of glass protein can be identified from large libraries of natural products, synthetic (or semi-synthetic) extracts, and chemical libraries using methods that are well known in the art. Those skilled in the field of drug discovery and development will understand that the precise source of test extracts or compounds is not critical to the screening methods of the invention and that dereplication, or the elimination of replicates or repeats of materials already known for their therapeutic activities for heart disease can be employed whenever possible.

Candidate compounds to be tested include purified (or substantially purified) molecules or one or more component of a mixture of compounds (e.g., an extract or supernatant obtained from cells; Ausubel et al., supra) and such compounds further include both naturally occurring or artificially derived chemicals and modifications of existing compounds. For example, candidate compounds can be polypeptides, synthesized organic or inorganic molecules, naturally occurring organic or inorganic molecules, nucleic acid molecules, and components thereof.

Numerous sources of naturally occurring candidate compounds are readily available to those skilled in the art. For example, naturally occurring compounds can be found in cell (including plant, fungal, prokaryotic, and animal) extracts, mammalian serum, growth medium in which mammalian cells have been cultured, protein expression libraries, or fermentation broths. In addition, libraries of natural compounds in the form of bacterial, fungal, plant, and animal extracts are commercially available from a number of sources, including Biotics (Sussex, UK), Xenova (Slough, UK), Harbor Branch Oceanographic Institute (Ft. Pierce, Fla.), and PharmaMar, U.S.A. (Cambridge, Mass.). Furthermore, libraries of natural compounds can be produced, if desired, according to methods that are known in the art, e.g., by standard extraction and fractionation.

Artificially derived candidate compounds are also readily available to those skilled in the art. Numerous methods are available for generating random or directed synthesis (e.g., semi-synthesis or total synthesis) of any number of chemical compounds, including, for example, saccharide-, lipid-, peptide-, and nucleic acid molecule-based compounds. In addition, synthetic compound libraries are commercially available from Brandon Associates (Merrimack, N.H.) and Aldrich Chemicals (Milwaukee, Wis.). Libraries of synthetic compounds can also be produced, if desired, according to methods known in the art, e.g., by standard extraction and fractionation. Furthermore, if desired, any library or compound can be readily modified using standard chemical, physical, or biochemical methods.

When a crude extract is found to have an effect on the development or persistence of heart disease, further fractionation of the positive lead extract can be carried out to isolate chemical constituents responsible for the observed effect. Thus, the goal of the extraction, fractionation, and purification process is the careful characterization and identification of a chemical entity within the crude extract having a desired activity. The same assays described herein for the detection of activities in mixtures of compounds can be used to purify the active component and to test derivatives of these compounds. Methods of fractionation and purification of such heterogeneous extracts are well known in the art. If desired, compounds shown to be useful agents for treatment can be chemically modified according to methods known in the art.

### Animal Model Systems

The invention also provides animal model systems for use in carrying out the screening methods described above. Examples of these model systems include zebrafish and other animals, such as mice, that have a mutation (e.g., the heart of glass mutation) in a gene encoding the heart of glass protein. For example, a zebrafish model that can be used in the invention can include a mutation that results in a lack of heart of glass protein production or production of a truncated (e.g., by introduction of a stop codon) or otherwise altered heart of glass gene product. As a specific example, a zebrafish having the heart of glass mutation can be used (see below).

### Treatment or Prevention of Heart Failure

Compounds identified using the screening methods described above can be used to treat patients that have or are at risk of developing heart disease, such as heart failure. Nucleic acid molecules encoding the heart of glass protein, as well as these proteins themselves, can also be used in such methods. Treatment may be required only for a short period of time or may, in some form, be required throughout a patient's lifetime. Any continued need for treatment, however, can be determined using, for example, the diagnostic methods described above. In considering various therapies, it is to be understood that such therapies are, preferably, targeted to the affected or potentially affected organ (i.e., the heart).

Treatment or prevention of diseases resulting from a mutated gene encoding the heart of glass protein can be accomplished, for example, by modulating the function of a mutant heart of glass protein. Treatment can also be accomplished by delivering normal heart of glass protein to appropriate cells, altering the levels of normal or mutant heart of glass protein, replacing a mutant gene encoding a heart of glass protein with a normal gene encoding the heart of glass protein, or administering a normal gene encoding the heart of glass protein. It is also possible to correct the effects of a defect in a gene encoding the heart of glass protein by modifying the physiological pathway (e.g., a signal transduction pathway) in which the heart of glass protein participates.

In a patient diagnosed as being heterozygous for a gene encoding a mutant heart of glass protein, or as susceptible to such mutations or aberrant heart of glass expression (even if those mutations or expression patterns do not yet result in alterations in expression or biological activity of the heart of glass), any of the therapies described herein can be administered before the occurrence of the disease phenotype. In particular, compounds shown to have an effect on the phenotype of mutants, or to modulate expression of heart of glass proteins can be administered to patients diagnosed with potential or actual heart disease by any standard dosage and route of administration.

Any appropriate route of administration can be employed to administer a compound found to be effective in treating or preventing heart failure according to the invention. For example, administration can be parenteral, intravenous, intra-arterial, subcutaneous, intramuscular, intraventricular, intracapsular, intraspinal, intracisternal, intraperitoneal, intranasal, by aerosol, by suppository, or oral.

A therapeutic compound of the invention can be administered within a pharmaceutically-acceptable diluent, carrier, or excipient, in unit dosage form. Administration can begin before or after the patient is symptomatic. Methods that are well known in the art for making formulations are found, for example, in Remington's Pharmaceutical Sciences (18^{th} edition), ed. A. Gennaro, 1990, Mack Publishing Company, Easton, Pa. Therapeutic formulations can be in the form of liquid solutions or suspensions. Formulations for parenteral administration can, for example, contain excipients; sterile water; or saline; polyalkylene glycols, such as polyethylene glycol; oils of vegetable origin; or hydrogenated napthalenes. Biocompatible, biodegradable lactide polymer, lactide/glycolide copolymer, or polyoxyethylene-polyoxypropylene copolymers can be used to control the release of the compounds. Other potentially useful parenteral delivery systems for compounds identified using the methods of the invention include ethylene-vinyl acetate copolymer particles, osmotic pumps, implantable infusion systems, and liposomes. For oral administration, formulations can be in the form of tablets or capsules. Formulations for inhalation can contain excipients, for example, lactose, or can be aqueous solutions containing, for example, polyoxyethylene-9-lauryl ether, glycocholate, and deoxycholate, or can be oily solutions for administration in the form of nasal drops, or as a gel. Alternatively, intranasal formulations can be in the form of powders or aerosols.

To replace a mutant protein with normal protein, or to add protein to cells that do not express sufficient or normal heart of glass protein, it may be necessary to obtain large amounts of pure heart of glass protein from cultured cell systems in which the protein is expressed (see, e.g., below). Delivery of the protein to the affected tissue can then be accomplished using appropriate packaging or administration systems.

Gene therapy is another therapeutic approach for preventing or ameliorating diseases (e.g., heart failure) caused by heart of glass gene defects. Nucleic acid molecules encoding wild type heart of glass protein can be delivered to cells that lack sufficient, normal heart of glass protein biological activity (e.g., cells carrying mutations (e.g., the heart of glass mutation) in heart of glass genes). The nucleic acid molecules must be delivered to those cells in a form in which they can be taken up by the cells and so that sufficient levels of protein, to provide effective heart of glass protein function, can be produced. Alternatively, for some heart of glass mutations, it may be possible slow the progression of the resulting disease or to modulate heart of glass protein activity by introducing another copy of a homologous gene bearing a second mutation in that gene, to alter the mutation, or to use another gene to block any negative effect.

Transducing retroviral, adenoviral, and adeno-associated viral vectors can be used for somatic cell gene therapy, especially because of their high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). For example, the full length heart of glass gene, or a portion thereof, can be cloned into a retroviral vector and expression can be driven from its endogenous promoter, from the retroviral long terminal repeat, or from a promoter specific for a target cell type of interest (such as cardiac muscle or other vascular cells). Other viral vectors that can be used include, for example, vaccinia virus, bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; Le Gal La Salle et al., Science 259:988-990, 1993; and Johnson, Chest 107:77 S-83S, 1995). Retroviral vectors are particularly well developed and have been used in clinical settings (Rosenberg et al., N. Engl. J. Med 323:370, 1990; Anderson et al., U.S. Pat. No. 5,399,346).

Non-viral approaches can also be employed for the introduction of therapeutic DNA into cells predicted to be subject to diseases involving the heart of glass protein. For example, a heart of glass nucleic acid molecule or an antisense nucleic acid molecule can be introduced into a cell by lipofection (Felgner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990).

Gene transfer can also be achieved using non-viral means involving transfection in vitro. Such methods include use of calcium phosphate, DEAE dextran, electroporation, and protoplast fusion. Liposomes can also be potentially beneficial for delivery of DNA into a cell. Transplantation of normal genes into the affected tissues of a patient can also be accomplished by transferring a normal heart of glass protein into a cultivatable cell type ex vivo, after which the cell (or its descendants) are injected into a targeted tissue.

Heart of glass cDNA expression for use in gene therapy methods can be directed from any suitable promoter (e.g., the human cytomegalovirus (CMV), simian virus 40 (SV40), or metallothionein promoters), and regulated by any appropriate mammalian regulatory element. For example, if desired, enhancers known to preferentially direct gene expression in specific cell types can be used to direct heart of glass expression. The enhancers used can include, without limitation, those that are characterized as tissue- or cell-specific enhancers. Alternatively, if a heart of glass genomic clone is used as a therapeutic construct (such clones can be identified by hybridization with heart of glass cDNA, as described herein), regulation can be mediated by the cognate regulatory sequences or, if desired, by regulatory sequences derived from a heterologous source, including any of the promoters or regulatory elements described above.

Antisense-based strategies can be employed to explore heart of glass protein gene function and as a basis for therapeutic drug design. These strategies are based on the principle that sequence-specific suppression of gene expression (via transcription or translation) can be achieved by intracellular hybridization between genomic DNA or mRNA and a complementary antisense species. The formation of a hybrid RNA duplex interferes with transcription of the target heart of glass protein-encoding genomic DNA molecule, or processing, transport, translation, or stability of the target heart of glass mRNA molecule.

Antisense strategies can be delivered by a variety of approaches. For example, antisense oligonucleotides or antisense RNA can be directly administered (e.g., by intravenous injection) to a subject in a form that allows uptake into cells. Alternatively, viral or plasmid vectors that encode antisense RNA (or antisense RNA fragments) can be introduced into a cell in vivo or ex vivo. Antisense effects can be induced by control (sense) sequences; however, the extent of phenotypic changes is highly variable. Phenotypic effects induced by antisense effects are based on changes in criteria such as protein levels, protein activity measurement, and target mRNA levels.

Heart of glass gene therapy can also be accomplished by direct administration of antisense heart of glass mRNA to a cell that is expected to be adversely affected by the expression of wild type or mutant heart of glass protein. The antisense heart of glass mRNA can be produced and isolated by any standard technique, but is most readily produced by in vitro transcription using an antisense heart of glass cDNA under the control of a high efficiency promoter (e.g., the T7 promoter). Administration of antisense heart of glass mRNA to cells can be carried out by any of the methods for direct nucleic acid molecule administration described above.

An alternative strategy for inhibiting heart of glass protein function using gene therapy involves intracellular expression of an anti-heart of glass protein antibody or a portion of an anti-heart of glass protein antibody. For example, the gene (or gene fragment) encoding a monoclonal antibody that specifically binds to a heart of glass protein and inhibits its biological activity can be placed under the transcriptional control of a tissue-specific gene regulatory sequence.

Another therapeutic approach included in the invention involves administration of a recombinant heart of glass polypeptide, either directly to the site of a potential or actual disease-affected tissue (for example, by injection) or systemically (for example, by any conventional recombinant protein administration technique). The dosage of the heart of glass protein depends on a number of factors, including the size and health of the individual patient but, generally, between 0.1 mg and 100 mg, inclusive, is administered per day to an adult in any pharmaceutically acceptable formulation.

### Synthesis of Heart of Glass Proteins, Polypeptides, and Polypeptide Fragments

Those skilled in the art of molecular biology will understand that a wide variety of expression systems can be used to produce the recombinant heart of glass proteins. As discussed further below, the precise host cell used is not critical to the invention. The heart of glass proteins can be produced in a prokaryotic host (e.g., E. coli) or in a eukaryotic host (e.g., S. cerevisiae, insect cells such as Sf9 cells, or mammalian cells such as COS-1, NIH 3T3, or HeLa cells). These cells are commercially available from, for example, the American Type Culture Collection, Manassas, Va. (see also Ausubel et al., supra). The method of transformation and the choice of expression vehicle (e.g., expression vector) will depend on the host system selected. Transformation and transfection methods are described, e.g., in Ausubel et al., supra, and expression vehicles can be chosen from those provided, e.g., in Pouwels et al., Cloning Vectors: A Laboratory Manual, 1985, Supp. 1987. Specific examples of expression systems that can be used in the invention are described further, as follows.

For protein expression, eukaryotic or prokaryotic expression systems can be generated in which heart of glass gene sequences are introduced into a plasmid or other vector, which is then used to transform living cells. Constructs in which full-length heart of glass cDNAs, containing the entire open reading frame, inserted in the correct orientation into an expression plasmid can be used for protein expression. Alternatively, portions of heart of glass gene sequences, including wild type or mutant heart of glass sequences, can be inserted. Prokaryotic and eukaryotic expression systems allow various important functional domains of heart of glass proteins to be recovered, if desired, as fusion proteins, and then used for binding, structural, and functional studies, and also for the generation of antibodies.

Typical expression vectors contain promoters that direct synthesis of large amounts of mRNA corresponding to a nucleic acid molecule that has been inserted into the vector. They can also include a eukaryotic or prokaryotic origin of replication, allowing for autonomous replication within a host cell, sequences that confer resistance to an otherwise toxic drug, thus allowing vector-containing cells to be selected in the presence of the drug, and sequences that increase the efficiency with which the synthesized mRNA is translated. Stable long-term vectors can be maintained as freely replicating entities by using regulatory elements of, for example, viruses (e.g., the OriP sequences from the Epstein Barr Virus genome). Cell lines can also be produced that have the vector integrated into genomic DNA of the cells, and, in this manner, the gene product can be produced in the cells on a continuous basis.

Expression of foreign molecules in bacteria, such as Escherichia coli, requires the insertion of a foreign nucleic acid molecule, e.g., a heart of glass nucleic acid molecule, into a bacterial expression vector. Such plasmid vectors include several elements required for the propagation of the plasmid in bacteria, and for expression of foreign DNA contained within the plasmid. Propagation of only plasmid-bearing bacteria is achieved by introducing, into the plasmid, a selectable marker-encoding gene that allows plasmid-bearing bacteria to grow in the presence of an otherwise toxic drug. The plasmid also contains a transcriptional promoter capable of directing synthesis of large amounts of mRNA from the foreign DNA. Such promoters can be, but are not necessarily, inducible promoters that initiate transcription upon induction by culture under appropriate conditions (e.g., in the presence of a drug that activates the promoter). The plasmid also, preferably, contains a polylinker to simplify insertion of the gene in the correct orientation within the vector.

Once an appropriate expression vector containing a heart of glass gene, or a fragment, fusion, or mutant thereof, is constructed, it can be introduced into an appropriate host cell using a transformation technique, such as, for example, calcium phosphate transfection, DEAE-dextran transfection, electroporation, microinjection, protoplast fusion, or liposome-mediated transfection. Host cells that can be transfected with the vectors of this invention can include, but are not limited to, E. coli or other bacteria, yeast, fungi, insect cells (using, for example, baculoviral vectors for expression), or cells derived from mice, humans, or other animals. Mammalian cells can also be used to express heart of glass proteins using a virus expression system (e.g., a vaccinia virus expression system) described, for example, in Ausubel et al., supra.

In vitro expression of heart of glass proteins, fusions, polypeptide fragments, or mutants encoded by cloned DNA can also be carried out using the T7 late-promoter expression system. This system depends on the regulated expression of T7 RNA polymerase, an enzyme encoded in the DNA of bacteriophage T7. The T7 RNA polymerase initiates transcription at a specific 23-bp promoter sequence called the T7 late promoter. Copies of the T7 late promoter are located at several sites on the T7 genome, but none are present in E. coli chromosomal DNA. As a result, in T7-infected E. Coli, T7 RNA polymerase catalyzes transcription of viral genes, but not E. coli genes. In this expression system, recombinant E. coli cells are first engineered to carry the gene encoding T7 RNA polymerase next to the lac promoter. In the presence of IPTG, these cells transcribe the T7 polymerase gene at a high rate and synthesize abundant amounts of T7 RNA polymerase. These cells are then transformed with plasmid vectors that carry a copy of the T7 late promoter protein. When IPTG is added to the culture medium containing these transformed E. coli cells, large amounts of T7 RNA polymerase are to produced. The polymerase then binds to the T7 late promoter on the plasmid expression vectors, catalyzing transcription of the inserted cDNA at a high rate. Since each E. coli cell contains many copies of the expression vector, large amounts of mRNA corresponding to the cloned cDNA can be produced in this system and the resulting protein can be radioactively labeled.

Plasmid vectors containing late promoters and the corresponding RNA polymerases from related bacteriophages, such as T3, T5, and SP6, can also be used for in vitro production of proteins from cloned DNA. E. coli can also be used for expression using an M13 phage, such as mGPI-2. Furthermore, vectors that contain phage lambda regulatory sequences, or vectors that direct the expression of fusion proteins, for example, a maltose-binding protein fusion protein or a glutathione-S-transferase fusion protein, also can be used for expression in E. coli.

Eukaryotic expression systems are useful for obtaining appropriate post-translational modification of expressed proteins. Transient transfection of a eukaryotic expression plasmid containing a heart of glass protein into a eukaryotic host cell allows the transient production of a heart of glass protein by the transfected host cell. Heart of glass proteins can also be produced by a stably-transfected eukaryotic (e.g., mammalian) cell line. A number of vectors suitable for stable transfection of mammalian cells are available to the public (see, e.g., Pouwels et al., supra), as are methods for constructing lines including such cells (see, e.g., Ausubel et al., supra).

In one example, cDNA encoding a heart of glass protein, fusion, mutant, or polypeptide fragment is cloned into an expression vector that includes the dihydrofolate reductase (DHFR) gene. Integration of the plasmid and, therefore, integration of the heart of glass protein-encoding gene, into the host cell chromosome is selected for by inclusion of 0.01-300 μM methotrexate in the cell culture medium (Ausubel et al., supra). This dominant selection can be accomplished in most cell types. Recombinant protein expression can be increased by DHFR-mediated amplification of the transfected gene. Methods for selecting cell lines bearing gene amplifications are described in Ausubel et al., supra. These methods generally involve extended culture in medium containing gradually increasing levels of methotrexate. The most commonly used DHFR-containing expression vectors are pCVSEII-DHFR and pAdD26SV(A) (described, for example, in Ausubel et al., supra). The host cells described above or, preferably, a DHFR-deficient CHO cell line (e.g., CHO DHFR-cells, ATCC Accession No. CRL 9096) are among those that are most preferred for DHFR selection of a stably transfected cell line or DHFR-mediated gene amplification.

Another preferred eukaryotic expression system is the baculovirus system using, for example, the vector pBacPAK9, which is available from Clontech (Palo Alto, Calif.). If desired, this system can be used in conjunction with other protein expression techniques, for example, the myc tag approach described by Evan et al. (Molecular and Cellular Biology 5:3610-3616, 1985).

Once a recombinant protein is expressed, it can be isolated from the expressing cells by cell lysis followed by protein purification techniques, such as affinity chromatography. In this example, an anti-heart of glass protein antibody, which can be produced by the methods described herein, can be attached to a column and used to isolate the recombinant heart of glass proteins. Lysis and fractionation of heart of glass protein-harboring cells prior to affinity chromatography can be performed by standard methods (see, e.g., Ausubel et al., supra). Once isolated, the recombinant protein can, if desired, be purified further by, e.g., high performance liquid chromatography (HPLC; e.g., see Fisher, Laboratory Techniques In Biochemistry and Molecular Biology, Work and Burdon, Eds., Elsevier, 1980).

Polypeptides of the invention, particularly short heart of glass protein fragments and longer fragments of the N-terminus and C-terminus of the heart of glass protein, can also be produced by chemical synthesis (e.g., by the methods described in Solid Phase Peptide Synthesis, 2^{nd} ed., 1984, The Pierce Chemical Co., Rockford, Ill.). These general techniques of polypeptide expression and purification can also be used to produce and isolate useful heart of glass protein fragments or analogs, as described herein.

### Heart of Glass Protein Fragments

Polypeptide fragments that include various portions of heart of glass proteins are useful in identifying the domains of the heart of glass protein that are important for its biological activities, such as protein-protein interactions and transcription. Methods for generating such fragments are well known in the art (see, for example, Ausubel et al., supra), using the nucleotide sequences provided herein. For example, a heart of glass protein fragment can be generated by PCR amplifying a desired heart of glass protein nucleic acid molecule fragment using oligonucleotide primers designed based upon heart of glass nucleic acid sequences. Preferably, the oligonucleotide primers include unique restriction enzyme sites that facilitate insertion of the amplified fragment into the cloning site of an expression vector (e.g., a mammalian expression vector, see above). This vector can then be introduced into a cell (e.g., a mammalian cell; see above) by artifice, using any of the various techniques that are known in the art, such as those described herein, resulting in the production of a heart of glass protein fragment in the cell containing the expression vector. Heart of glass protein fragments (e.g., chimeric fusion proteins) can also be used to raise antibodies specific for various regions of the heart of glass protein using, for example, the methods described below.

### Heart of Glass Protein Antibodies

To prepare polyclonal antibodies, heart of glass proteins, fragments of heart of glass proteins, or fusion proteins containing defined portions of heart of glass proteins can be synthesized in, e.g., bacteria by expression of corresponding DNA sequences contained in a suitable cloning vehicle. Fusion proteins are commonly used as a source of antigen for producing antibodies. Two widely used expression systems for E. coli are lacZ fusions using the pUR series of vectors and trpE fusions using the pATH vectors. The proteins can be purified, coupled to a carrier protein, mixed with Freund's adjuvant to enhance stimulation of the antigenic response in an inoculated animal, and injected into rabbits or other laboratory animals. Alternatively, protein can be isolated from heart of glass protein-expressing cultured cells. Following booster injections at bi-weekly intervals, the rabbits or other laboratory animals are then bled and the sera isolated. The sera can be used directly or can be purified prior to use by various methods, including affinity chromatography employing reagents such as Protein A-Sepharose, antigen-Sepharose, and anti-mouse-Ig-Sepharose. The sera can then be used to probe protein extracts from heart of glass protein-expressing tissue fractionated by polyacrylamide gel electrophoresis to identify heart of glass proteins. Alternatively, synthetic peptides can be made that correspond to antigenic portions of the protein and used to inoculate the animals.

To generate peptide or full-length protein for use in making, for example, heart of glass protein-specific antibodies, a heart of glass protein coding sequence can be expressed as a C-terminal or N-terminal fusion with glutathione S-transferase (GST; Smith et al., Gene 67:31-40, 1988). The fusion protein can be purified on glutathione-Sepharose beads, eluted with glutathione, cleaved with a protease, such as thrombin or Factor-Xa (at the engineered cleavage site), and purified to the degree required to successfully immunize rabbits. Primary immunizations can be carried out with Freund's complete adjuvant and subsequent immunizations performed with Freund's incomplete adjuvant. Antibody titers can be monitored by Western blot and immunoprecipitation analyses using the protease-cleaved heart of glass protein fragment of the GST-heart of glass protein. Immune sera can be affinity purified using CNBr-Sepharose-coupled heart of glass protein. Antiserum specificity can be determined using a panel of unrelated GST fusion proteins.

Alternatively, monoclonal heart of glass protein antibodies can be produced by using, as an antigen, heart of glass protein isolated from heart of glass protein-expressing cultured cells or heart of glass protein isolated from tissues. The cell extracts, or recombinant protein extracts containing heart of glass protein, can, for example, be injected with Freund's adjuvant into mice. Several days after being injected, the mouse spleens can be removed, the tissues disaggregated, and the spleen cells suspended in phosphate buffered saline (PBS). The spleen cells serve as a source of lymphocytes, some of which would be producing antibody of the appropriate specificity. These can then be fused with permanently growing myeloma partner cells, and the products of the fusion plated into a number of tissue culture wells in the presence of selective agents, such as hypoxanthine, aminopterine, and thymidine (HAT). The wells can then be screened by ELISA to identify those containing cells making antibody capable of binding to a heart of glass protein, polypeptide fragment, or mutant thereof. These cells can then be re-plated and, after a period of growth, the wells containing these cells can be screened again to identify antibody-producing cells. Several cloning procedures can be carried out until over 90% of the wells contain single clones that are positive for specific antibody production. From this procedure, a stable line of clones that produce the antibody can be established. The monoclonal antibody can then be purified by affinity chromatography using Protein A Sepharose and ion exchange chromatography, as well as variations and combinations of these techniques. Once produced, monoclonal antibodies are also tested for specific heart of glass protein recognition by Western blot or immunoprecipitation analysis (see, e.g., Kohler et al., Nature 256:495, 1975; Kohler et al., European Journal of Immunology 6:511, 1976; Kohler et al., European Journal of Immunology 6:292, 1976; Hammerling et al., In Monoclonal Antibodies and T Cell Hybridomas, Elsevier, New York, N.Y., 1981; Ausubel et al., supra).

As an alternate or adjunct immunogen to GST fusion proteins, peptides corresponding to relatively unique hydrophilic regions of the heart of glass protein can be generated and coupled to keyhole limpet hemocyanin (KLH) through an introduced C-terminal lysine. Antiserum to each of these peptides can be similarly affinity-purified on peptides conjugated to BSA, and specificity tested by ELISA and Western blotting using peptide conjugates, and by Western blotting and immunoprecipitation using the heart of glass protein, for example, expressed as a GST fusion protein.

Antibodies of the invention can be produced using heart of glass protein amino acid sequences that do not reside within highly conserved regions, and that appear likely to be antigenic, as analyzed by criteria such as those provided by the Peptide Structure Program (Genetics Computer Group Sequence Analysis Package, Program Manual for the GCG Package, Version 7, 1991) using the algorithm of Jameson et al., CABIOS 4:181, 1988. These fragments can be generated by standard techniques, e.g., by PCR, and cloned into the pGEX expression vector. GST fusion proteins can be expressed in E. coli and purified using a glutathione-agarose affinity matrix (Ausubel et al., supra). To generate rabbit polyclonal antibodies, and to minimize the potential for obtaining antisera that is non-specific, or exhibits low-affinity binding to a heart of glass protein, two or three fusions are generated for each protein, and each fusion is injected into at least two rabbits. Antisera are raised by injections in series, preferably including at least three booster injections.

In addition to intact monoclonal and polyclonal anti-heart of glass protein antibodies, the invention features various genetically engineered antibodies, humanized antibodies, and antibody fragments, including F(ab′)2, Fab′, Fab, Fv, and sFv fragments. Truncated versions of monoclonal antibodies, for example, can be produced by recombinant methods in which plasmids are generated that express the desired monoclonal antibody fragment(s) in a suitable host. Antibodies can be humanized by methods known in the art, e.g., monoclonal antibodies with a desired binding specificity can be commercially humanized (Scotgene, Scotland; Oxford Molecular, Palo Alto, Calif.). Fully human antibodies, such as those expressed in transgenic animals, are also included in the invention (Green et al., Nature Genetics 7:13-21, 1994).

Ladner (U.S. Pat. Nos. 4,946,778 and 4,704,692) describes methods for preparing single polypeptide chain antibodies. Ward et al., Nature 341:544-546, 1989, describes the preparation of heavy chain variable domains, which they term “single domain antibodies,” and which have high antigen-binding affinities. McCafferty et al., Nature 348:552-554, 1990, show that complete antibody V domains can be displayed on the surface of fd bacteriophage, that the phage bind specifically to antigen, and that rare phage (one in a million) can be isolated after affinity chromatography. Boss et al., U.S. Pat. No. 4,816,397, describes various methods for producing immunoglobulins, and immunologically functional fragments thereof, that include at least the variable domains of the heavy and light chains in a single host cell. Cabilly et al., U.S. Pat. No. 4,816,567, describes methods for preparing chimeric antibodies.

### Use of Heart of Glass Antibodies

Antibodies to heart of glass proteins can be used, as noted above, to detect heart of glass proteins or to inhibit the biological activities of heart of glass proteins. For example, a nucleic acid molecule encoding an antibody or portion of an antibody can be expressed within a cell to inhibit heart of glass protein function. In addition, the antibodies can be coupled to compounds, such as radionuclides and liposomes, for diagnostic or therapeutic uses. Antibodies that specifically recognize extracellular domains of heart of glass proteins are useful for targeting such attached moieties to cells displaying such heart of glass protein domains at their surfaces. Antibodies that inhibit the activity of a heart of glass polypeptide described herein can also be useful in preventing or slowing the development of a disease caused by inappropriate expression of a wild type or mutant heart of glass gene.

### Detection of Heart of Glass Gene Expression

As noted, the antibodies described above can be used to monitor heart of glass protein expression. In situ hybridization of RNA can be used to detect the expression of heart of glass genes. RNA in situ hybridization techniques rely upon the hybridization of a specifically labeled nucleic acid probe to the cellular RNA in individual cells or tissues. Therefore, RNA in situ hybridization is a powerful approach for studying tissue- and temporal-specific gene expression. In this method, oligonucleotides, cloned DNA fragments, or antisense RNA transcripts of cloned DNA fragments corresponding to unique portions of heart of glass genes are used to detect specific mRNA species, e.g., in the tissues of animals, such as mice, at various developmental stages. Other gene expression detection techniques are known to those of skill in the art and can be employed for detection of heart of glass gene expression.

### Identification of Additional Heart of Glass Genes

Standard techniques, such as the polymerase chain reaction (PCR) and DNA hybridization, can be used to clone heart of glass homologues in other species and heart of glass-related genes in humans. Heart of glass-related genes and homologues can be readily identified using low-stringency DNA hybridization or low-stringency PCR with human heart of glass probes or primers. Degenerate primers encoding human heart of glass or human heart of glass-related amino acid sequences can be used to clone additional heart of glass-related genes and homologues by RT-PCR.

### Construction of Transgenic Animals and Knockout Animals

Characterization of heart of glass genes provides information that allows heart of glass knockout animal models to be developed by homologous recombination. Preferably, a heart of glass knockout animal is a mammal, most preferably a mouse. Similarly, animal models of heart of glass overproduction can be generated by integrating one or more heart of glass sequences into the genome of an animal, according to standard transgenic techniques. Moreover, the effect of heart of glass mutations (e.g., dominant gene mutations) can be studied using transgenic mice carrying mutated heart of glass transgenes or by introducing such mutations into the endogenous heart of glass gene, using standard homologous recombination techniques.

A replacement-type targeting vector, which can be used to create a knockout model, can be constructed using an isogenic genomic clone, for example, from a mouse strain such as 129/Sv (Stratagene Inc., LaJolla, Calif.). The targeting vector can be introduced into a suitably derived line of embryonic stem (ES) cells by electroporation to generate ES cell lines that carry a profoundly truncated form of heart of glass gene. To generate chimeric founder mice, the targeted cell lines are injected into a mouse blastula-stage embryo. Heterozygous offspring can be interbred to homozygosity. Heart of glass knockout mice provide a tool for studying the role of heart of glass in embryonic development and in disease. Moreover, such mice provide the means, in vivo, for testing therapeutic compounds for amelioration of diseases or conditions involving heart of glass-dependent or a heart of glass-affected pathway.

### Experimental Results

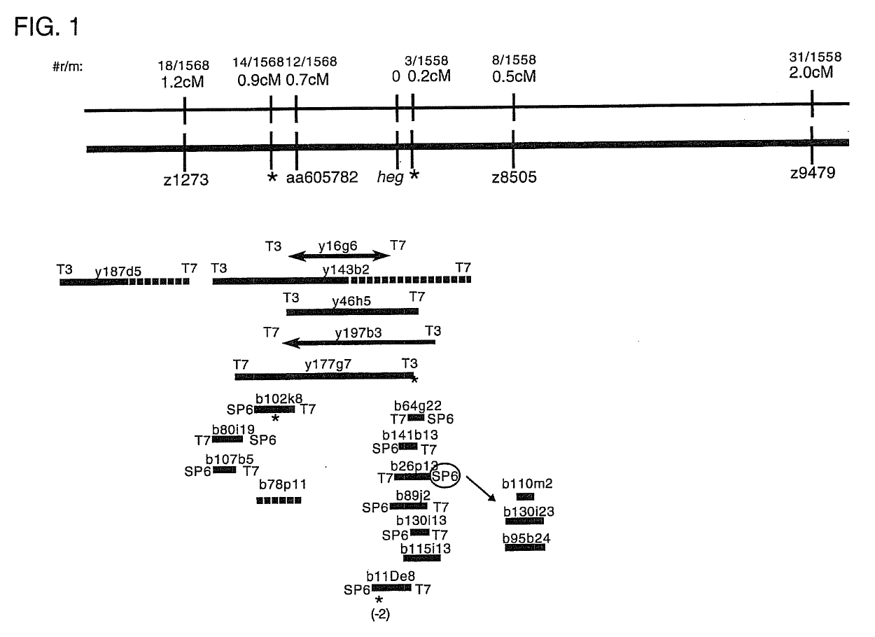

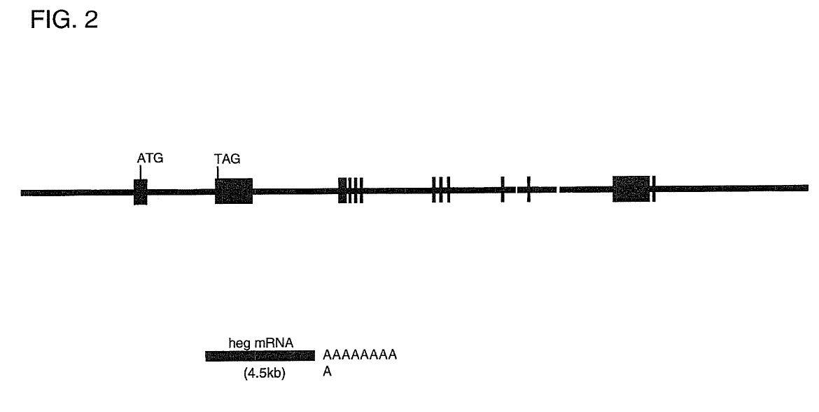

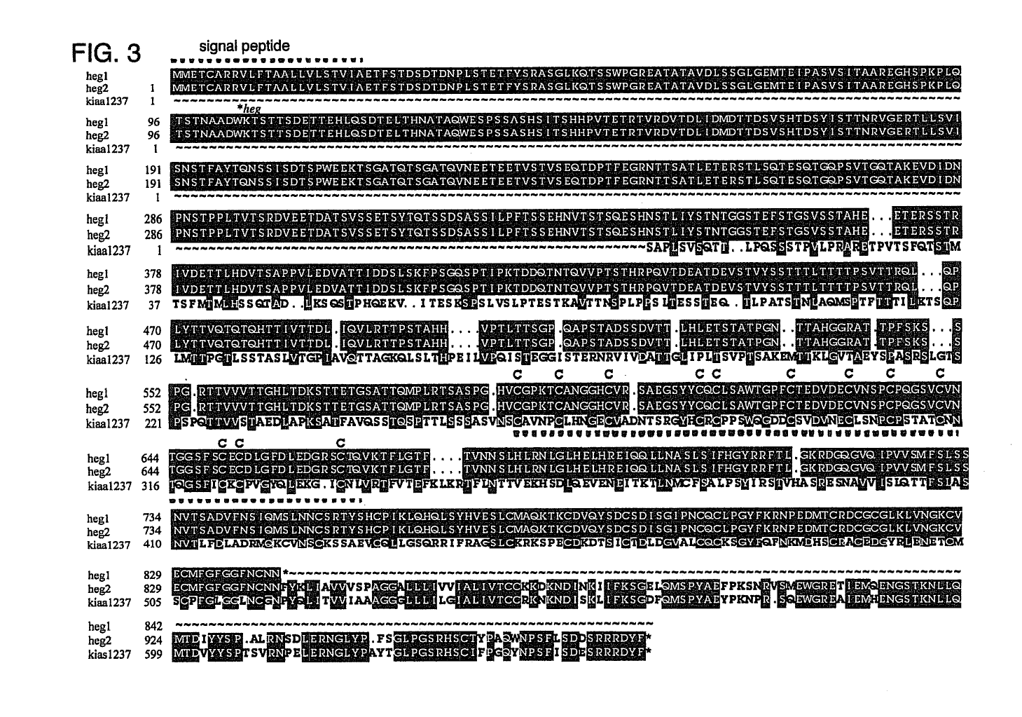

To better understand the basis of the heart of glass (heg) mutation, we initiated positional cloning projects and have determined the gene responsible for the heg mutation (FIGS. 1 and 2). This gene is not dominated by any known structural motifs, but does possess two short peptide stretches that have the distinctive six cysteines associated with EGF-repeats (amino acids 580 to 660), which are found in many different proteins of diverse function. As shown in FIG. 3, the protein exhibits about 34% identity over the deduced 650 amino acids of the EST KIAA1237 (Genbank entry BAA86551.1), and about 80% identity over the C-terminal 100 amino acids.

The mutation in the heart of glass gene is a stop codon resulting from a G to A change at residue 497, switching a tryptophan codon to a stop codon (TGG to TAG). This mutation occurs at amino acid 103, early in the sequence, and would result in a dramatic protein truncation. A cleavable signal peptide is predicted based on the primary structure, with a possible cleavage site between amino acids 23 and 24. This structure would still be encoded by the truncated protein and possibly cleaved, but the resulting peptide of 80 amino acids may not have any physiological function.

To characterize this gene, we have examined the expression pattern in zebrafish embryos using whole mount in situ hybridization at several stages of development with the 3′ cDNA fragment isolated from a 24 hour cDNA library. The gene is expressed in the heart at 24 hours and 48 hours of development, but at other stages demonstrates a less restricted expression pattern. Further experiments indicate that beg is expressed in the outflow tract of the heart and ventricle, in the endocardium.

Additional experiments include injection experiments using morphlino antisense oligonucleotides, to phenocopy the mutation to provide further evidence that we have identified the mutated gene in beg embryos. Initial experiments reveal a loss of circulation in treated embryos, as well as substantial pericardial edema. In several embryos there is no hypertrophy, but there is edema and lack of circulation. The antisense oligonucleotide may not completely block translation of the heg message, while the stop codon in the heg mutants may completely prevent any functional beg protein from being synthesized. Thus, the phenotype associated with the morpholino injections may in fact be a hypermorphic response. Also, the phenotype is most obvious at 48 hours of development, by which time the oligonucleotide may be losing activity.

### OTHER EMBODIMENTS

All publications and patent applications mentioned in this specification are herein incorporated by reference to the same extent as if each independent publication or patent application was specifically and individually indicated to be incorporated by reference.

While the invention has been described in connection with specific embodiments thereof, it is to be understood that it is capable of further modifications and this application is intended to cover any variations, uses, or adaptations of the invention following, in general, the principles of the invention and including such departures from the present disclosure that come within known or customary practice within the art to which the invention pertains and can be applied to the essential features hereinbefore set forth, and follows in the scope of the appended claims.

What is claimed is:

1. A method of determining whether a test subject has, or is at risk of developing, a disease or condition related to a heart of glass protein, said method comprising analyzing a nucleic acid molecule of a sample from the test subject to determine whether the test subject has a mutation in a gene encoding said protein, wherein the presence of a mutation indicates that said test subject has, or is at risk of developing, a disease related to a heart of glass protein.

2. The method of claim 1, further comprising the step of using nucleic acid molecule primers specific for a gene encoding a heart of glass protein for nucleic acid molecule amplification of the gene by the polymerase chain reaction.

3. The method of claim 1, wherein determination of whether said gene comprises a mutation is carried out by sequencing a nucleic acid molecule encoding a heart of glass protein from said test subject.

4. The method of claim 1, wherein said test subject is a mammal.

5. The method of claim 1, wherein said test subject is a human.

6. The method of claim 1, wherein said disease or condition is heart disease.

7. The method of claim 1, wherein said disease or condition is heart failure.

8-17. (canceled)

18. A substantially pure zebrafish heart of glass polypeptide.

19. The polypeptide of claim 18, wherein said polypeptide comprises an amino acid sequence that is substantially identical to the amino acid sequence of SEQ ID NO:2 or SEQ ID NO:3.

20. The polypeptide of claim 19, wherein said polypeptide comprises the amino acid sequence of SEQ ID NO:2 or SEQ ID NO:3.

21. A substantially pure nucleic acid molecule comprising a sequence encoding a zebrafish heart of glass polypeptide of claim 18.

22. The nucleic acid molecule of claim 21, wherein said nucleic acid molecule encodes a polypeptide comprising an amino sequence that is substantially identical to the amino acid sequence of SEQ ID NO:2 or SEQ ID NO:3.

23. The nucleic acid molecule of claim 21, wherein said nucleic acid molecule encodes a polypeptide comprising the amino acid sequence of SEQ ID NO:2 or SEQ ID NO: 3.

24. The nucleic acid molecule of claim 21, wherein said nucleic acid molecules is DNA.

25. A vector comprising the nucleic acid molecule of claim 21.

26. A cell comprising the vector of claim 25.

27. A non-human transgenic animal comprising the nucleic acid molecule of claim 21.

28. The non-human transgenic animal of claim 27, wherein said animal is a zebrafish.

29. (canceled)

30. A cell from the non-human knockout animal of claim 27.

31-33. (canceled)

34. An antibody that specifically binds to a heart of glass polypeptide.

35-36. (canceled)
